(19) United States
(12) Patent Application Publication
(10) Pub. No.: US 2016/0312207 A1
(43) Pub. Date: Oct. 27, 2016

(54) R-SPONDIN ANTAGONISTS AND METHODS OF TREATING CANCER ASSOCIATED WITH ABERRANT ACTIVATION OF WNT SIGNALING

(71) Applicant: The Board of Trustees of the Leland Stanford Junior University, Palo Alto, CA (US)

(72) Inventors: Calvin J. Kuo, Palo Alto, CA (US); Kelley Yan, Los Altos Hills, CA (US); Junlei Chang, Menlo Park, CA (US)

(21) Appl. No.: 15/134,353

(22) Filed: Apr. 20, 2016

Related U.S. Application Data
(60) Provisional application No. 62/150,822, filed on Apr. 21, 2015.

(51) Int. Cl. C12N 9/00; A61K 9/00; A61K 38/53; C12N 7/00

(52) U.S. Cl. CPC C12N 9/93; C12N 7/00; A61K 9/0019; A61K 38/53; C12Y 603/02; C07K 2319/30; C12N 2710/10071; C12N 2710/10033; C07K 2319/33

(57) ABSTRACT

Compositions and methods for treating cancer associated with aberrant activation of the Wnt signaling pathway are disclosed. In particular, the invention relates to R-spondin antagonists comprising a soluble extracellular domain of ring finger 43 (RNF43) or E3 ubiquitin ligase zinc and ring finger 3 (ZNRF3) and methods of treating cancer with such antagonists.

### CROSS-REFERENCE TO RELATED APPLICATION

This application claims benefit under 35 U.S.C. §119(e) of provisional application 62/150,822, filed Apr. 21, 2015, which is hereby incorporated herein by reference in its entirety.

### STATEMENT REGARDING FEDERALLY SPONSORED RESEARCH OR DEVELOPMENT

This invention was made with Government support under contract DK085720 awarded by the National Institutes of Health. The Government has certain rights in the invention.

### TECHNICAL FIELD

The present invention pertains generally to compositions and methods of treating cancer associated with aberrant activation of the Wnt signaling pathway. In particular, the invention relates to R-spondin antagonists comprising a soluble extracellular domain of ring finger 43 (RNF43) or E3 ubiquitin ligase zinc and ring finger 3 (ZNRF3) and methods of treating cancer with such antagonists.

### BACKGROUND

R-spondins (RSpo) are a family of 4 secreted proteins that bear no structural similarity to Wnts, but strongly synergize with Wnts (>10-fold) to activate the canonical β-catenin/Tcf4 Wnt signaling (Kim et al. (2005) Science 309:1256-1259; Ootani et al. (2009) Nat Med 15:701-706). We previously demonstrated that R-spondins massively induce intestinal proliferation and expand Lgr5+ISC in vivo (Ootani et al., supra; Barry et al. (2013) Nature 493:106-110; Yan et al. (2012) Proc Natl Acad Sci USA 109:466-471). R-spondins1-4 are ligands for Lgr5 and the related receptors Lgr4 and Lgr6, which transduce signals amplifying Wnt signaling (Carmon et al. (2011) Proc Natl Acad Sci USA 108:11452-11457; Glinka et al. (2011) EMBO Rep 12:1055-1061; de Lau et al. (2011) Nature 476:293-297; Hsu et al. (2000) Mol Endocrinol 14:1257-1271). RSpo also acts via the transmembrane E3 ubiquitin ligase zinc and ring finger 3 (ZNRF3) and its homologue ring finger 43 (RNF43), which negatively regulate the Frizzled class of Wnt receptors, but upon RSpo binding to ZNRF3/RNF43, this negative regulation is inhibited, resulting in Frizzled up-regulation and increased Wnt signaling (Koo et al. (2012) Nature 488:665-669; Hao et al. (2012) Nature 485:195-200). Rspo proteins possess spatially distinct domains that allow their simultaneous engagement of receptors of both the Lgr4-6 class and the RNF43/ZNRF3 transmembrane E3 ubiquitin ligase family (Hao et al., supra; de Lau et al. (2014) Genes Dev 28:305-316; Xie et al. (2013) EMBO Rep 14(12):1120-1126; Peng et al. Cell Rep (2013) 3:1885-1892).

Colorectal cancer (CRC) is typically driven by Wnt pathway activation, with the TCGA describing that 85% of CRCs harbor APC mutations resulting in constitutive Wnt signaling. However, a recent report in Nature described RSpo2 or RSpo3 translocations in 10% of human colorectal cancers (CRC), resulting in greater than 80-fold upregulation of RSpo2/3 expression (Seshagiri et al. (2012) Nature 488:660-664). The translocations are mutually exclusive with APC mutations, indicating that RSpo translocation is an alternative mechanism for Wnt activation in CRC.

There remains a need for better methods of treating cancer associated with aberrant activation of the Wnt signaling pathway.

### SUMMARY

The present invention pertains generally to R-spondin antagonists and methods of treating cancer with such antagonists.

In one aspect, the invention includes an R-spondin antagonist comprising a soluble extracellular domain (ECD) of RNF43 or ZNRF3. In one embodiment, the R-spondin antagonist further comprises a signal peptide. In another embodiment, the R-spondin antagonist comprises residues corresponding to amino acids 1 to 192 of a RNF43 protein numbered relative to the reference sequence of SEQ ID NO:4.

In certain embodiments, the R-spondin antagonist comprises a soluble ECD of RNF43 comprising a polypeptide comprising the amino acid sequence of SEQ ID NO:2 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the R-spondin antagonist inhibits activation of Wnt signaling by an R-spondin.

In certain embodiments, the R-spondin antagonist is a fusion protein comprising an immunoglobulin Fc domain covalently linked to the soluble ECD of the RNF43 or ZNRF3. The immunoglobulin Fc fragment may be derived from an IgG (e.g., IgG1, IgG2, IgG3, or IgG4), IgM, IgE, IgA or IgD, or a combination or hybrid thereof. In one embodiment, the Fc fragment is derived from an IgG2a immunoglobulin, wherein the fragment comprises the amino acid sequence of SEQ ID NO:3 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the R-spondin antagonist inhibits activation of Wnt signaling by an R-spondin.

In another aspect, the invention includes a polynucleotide encoding an R-spondin antagonist described herein.

In another aspect, the invention includes a recombinant polynucleotide comprising a polynucleotide encoding an R-spondin antagonist operably linked to a promoter. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

In another aspect, the invention includes a host cell comprising a recombinant polynucleotide described herein.

In another aspect, the invention includes a method for producing an R-spondin antagonist, the method comprising: a) transforming a host cell with a recombinant polynucleotide described herein; b) culturing the transformed host cell under conditions whereby the R-spondin antagonist is expressed; and c) isolating the R-spondin antagonist from the host cell.

In another aspect, the invention includes a kit comprising an R-spondin antagonist described herein.

In another aspect, the invention includes a kit comprising a recombinant polynucleotide encoding an R-spondin antagonist described herein.

In another aspect, the invention includes a kit comprising a host cell transfected with a recombinant polynucleotide encoding an R-spondin antagonist described herein.

In another aspect, the invention includes a method of treating a subject for cancer, the method comprising administering a therapeutically effective amount of an R-spondin antagonist described herein to the subject.

In another aspect, the invention includes a method of treating a subject for cancer, the method comprising administering a therapeutically effective amount of a recombinant polynucleotide encoding an R-spondin antagonist described herein to the subject.

An R-spondin antagonist or a recombinant polynucleotide encoding it may be administered by any suitable mode of administration. In certain embodiments, the R-spondin antagonist or a recombinant polynucleotide encoding it is administered intravenously, intra-arterially, subcutaneously, or intralesionally to the subject. In another embodiment, the R-spondin antagonist or a recombinant polynucleotide encoding it is administered locally into a tumor of the subject. Multiple cycles of treatment may be administered to the subject for a time period sufficient to effect at least a partial tumor response or, more preferably, a complete tumor response.

These and other embodiments of the subject invention will readily occur to those of skill in the art in view of the disclosure herein.

### BRIEF DESCRIPTION OF THE FIGURES

FIG. 1 shows sequences of the native 23 amino acid signal peptide (SEQ ID NO:1), the RNF43 ectodomain (SEQ ID NO:2), and the mouse IgG Fc domain (SEQ ID NO:3).

FIGS. 2A and 2B show RSPO inhibition by a soluble RNF43 ectodomain (RNF43 ECD). FIG. 2A shows that Rspo proteins bind to both Lgr4-6 GPCR-like receptors and Rnf43/Znrf3 E3 ubiquitin ligases. RNF43/ZNRF3 degrade Frizzled and LRP Wnt receptor at baseline. But Rspo binding to RNF43/ZNRF3 inhibits their degradation of Frizzled and LRP Wnt receptors, which then accumulate and thus Wnt signaling is stimulated. FIG. 2B shows that the RNF43 ECD binds Rspo and prevents it from binding to RNF43/ZNRF3. Thus the RNF43/ZNRF3 degradation of Frizzled and LRP Wnt receptors is no longer suppressed. Accordingly, RNF43/ZNRF3 degradation of Frizzled and LRP Wnt receptors occurs in an unrestricted fashion, and Frizzed and LRP are decreased in abundance with an overall repression of Wnt signaling.

FIG. 3 shows an SDS-PAGE gel with a band at 48 kDa for the soluble recombinant RNF43-Fc ECD fusion protein, which was purified from the conditioned medium of transfected 293T cells to homogeneity using protein A agarose.

FIG. 4 shows a Biacore analysis of recombinant RNF43 ECD binding to human RSPO1-4 and comparison with the ZNRF3 ECD.

FIGS. 5A and 5B show that the RNF43-Fc ECD fusion protein potently neutralizes the ability of RSPO proteins to activate Wnt signaling in a TOP-FLASH Wnt reporter assay. FIG. 5A shows that the recombinant RNF43-Fc ECD fusion protein inhibits the ability of RSPO proteins to activate Wnt signaling in an L cell TOP-FLASH Wnt reporter assay. Wnt3a conditioned medium, recombinant human RSPO2 (500 ng/ml), Lgr5 ECD (100 μg/ml), RNF43 ECD (240 μg/ml), Fz8 CRD (50 μg/ml) were used. FIG. 5B shows that the transfected RNF43-Fc ECD fusion protein inhibits the ability of transfected RSPO2 and transfected Wnt3a to activate Wnt signaling in a 293 cell TOP-FLASH Wnt reporter assay.

FIGS. 6A-6D show that the RSPO3 fusion gene induces dysplasia in KRAS^{G12D}; p53^{-/-} colon organoids. FIG. 6A shows fusion cDNA. FIGS. 6B and 6C show lentiviral transduction of RSPO3 fusion and recipient KRAS^{G12D}; p53^{-/-} colon organoids. FIG. 6D shows that the RSPO3 fusion induces dysplasia (KRAS^{G12D}; p53^{-/-} colon organoids, d26 passage 2). Severe polypoid dysplasia is seen by H&E upon RSPO3 fusion transcript expression.

FIG. 7 shows that the Rspo3 fusion gene increases in vivo tumorigenicity and lung metastasis of Kras^{G12D}; p53^{-/-} colon organoids upon subcutaneous (s.c.) implantation into NOG mice.

FIGS. 8A and 8B show histology of the primary and metastatic tumors from KRAS^{G12D}; p53^{-/-} colon organoids with the RSPO3 fusion transcript revealed a common glandular histology. FIG. 8A shows a s.c. primary tumor. FIG. 8B shows lungs from KRAS^{G12D}; p53^{-/-} colon organoids with or without the RSPO3 fusion transcript.

FIG. 9 shows persistent circulating in vivo expression of the RNF43 ECD-Fc fusion by adenovirus after single i.v. injection.

FIG. 10 shows that soluble RNF43 ECD inhibits in vivo growth and metastasis of Rspo3 translocation-expressing colon organoid tumors.

FIG. 11 shows that RNF43 ECD ablates Lgr5+ intestinal stem cells in vivo without physiologic compromise or effects on intestinal architecture.

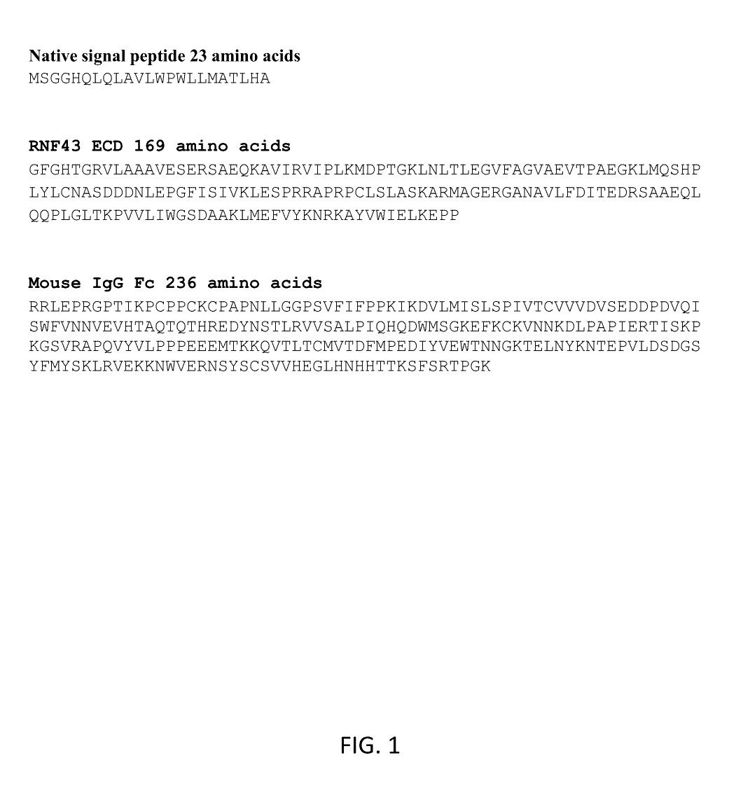

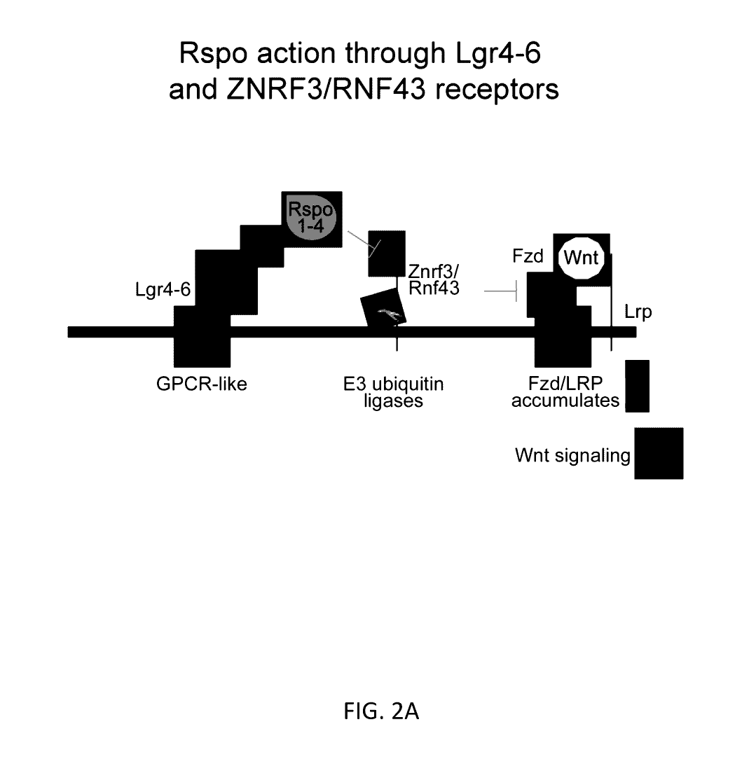

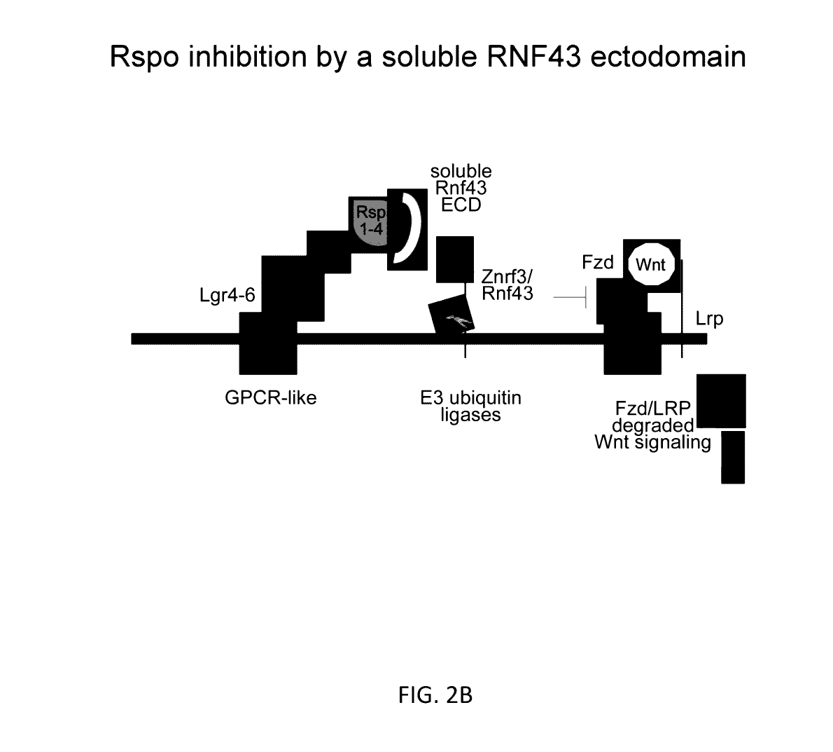

### DETAILED DESCRIPTION

The practice of the present invention will employ, unless otherwise indicated, conventional methods of medicine, chemistry, biochemistry, and molecular biology within the skill of the art. Such techniques are explained fully in the literature. See, e.g., S. P. Hoppler and R. T. Moon Wnt Signaling in Development and Disease: Molecular Mechanisms and Biological Functions (Wiley-Blackwell, 2014); Wnt Signaling (A Subject Collection from Cold Spring Harbor Perspectives in Biology, R. Nusse, X. He, and R. van Amerongen eds., Cold Spring Harbor Laboratory Press, 2012); Sambrook et al., Molecular Cloning: A Laboratory Manual (3^{rd} Edition, 2001); A. L. Lehninger, Biochemistry (Worth Publishers, Inc., current addition); Methods in Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an antagonist” includes two or more antagonists, and the like.

The term “about,” particularly in reference to a given quantity, is meant to encompass deviations of plus or minus five percent.

The terms “fusion protein,” “fusion polypeptide,” “RNF43 ECD-Fc fusion protein,” or “RNF43-Fc ECD” as used herein refer to a fusion comprising a signal peptide and extracellular domain (ECD) of an RNF43 protein in combination with an immunoglobulin Fc fragment as part of a single continuous chain of amino acids, which chain does not occur in nature. The RNF43 and Fc polypeptides may be connected directly to each other by peptide bonds or may be separated by intervening amino acid sequences. The fusion polypeptides may also contain sequences exogenous to the RNF43 and Fc polypeptides. For example, the fusion may include a linker, tag, or targeting sequence.

RNF43 nucleic acid and protein sequences may be derived from any source. A number of RNF43 nucleic acid and protein sequences are known. Representative RNF43 sequences are presented in SEQ ID NO:1 (signal peptide), SEQ ID NO:2 (ECD), and SEQ ID NO:4 (entire RNF43), and additional representative sequences are listed in the National Center for Biotechnology Information (NCBI) database. See, for example, NCBI entries: Accession Nos. NM_001305544, NM_001305545, NM_017763, NG_042894, NM_172448, NM_001135921, XM 006983219, XM 015961792, XM_007429040, XM_015880934, XM_015817188, XM_015646883, XM_015646881, XM_015607484, XM_006924877, XM_006924876, XM_006756201, XM_006756202, XM_015539550, XM_015495419, XM_005219941, XM_005583828, XM_015383684, XM_015383683, XM_006640990, XM_003642379, XM_015251931, XM_006219108, XM_015119152, XM_015098454, XM_012170327, XM_014989054, XM_015031841, XM_015088435, XM_014959717, XM_014881490, XM_014881488, XM_014826904, XM_014826896, XM_014826886, XM_014826878, XM_014826870, XM_014826861, XM_014826856, XM_014741472, XM_014741466, XM_014741463, XM_014741458, XM_014741453, XM_014741451, XM_014741448, XM_006267924, XM_006154644, XM_014529848, XM_005858364, XM_014529841, XM_006126750, XM_006126749, XM_014556497, XM_014556496, XM_006181416, XM_014526439, XM_014486261, XM_005900646, XM_014469427, XM_014461677, XM_006095618, XM_014461676, XM_008970573, XM_008970572, XM_008970571, XM_003817383, XM_008970569, XM_008970568, XM_008970567, XM_005425150, XM_005493759, XM_014255106, XM_005439600, XM_014136583, XM_011524956, XM_011524955, XM_011524954, XM_006532701, XM_011248844, XM_006536449, XM_011251825, XM_006247094, XM_008768041, XM_008768040, XM_006247092, XM_008768039, XM_006247091, XM_006247090, XM_008768038, XM_009432917, XM_003315636, XM_009432916, XM_009432915, XM_009432914, XM_009432913, XM_009432912, XR 675406, XM_014116703, XM_014116702, XM_014116701, XM_014116700, XM_014116699, XM_014116698, and XM_014116697; all of which sequences (as entered by the date of filing of this application) are herein incorporated by reference. Any of these sequences or a variant or fragment thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein comprising an RNF43 signal peptide and ECD, or a nucleic acid encoding such a fusion protein, as described herein.

The terms “polypeptide” and “protein” refer to a polymer of amino acid residues and are not limited to a minimum length. Thus, peptides, oligopeptides, dimers, multimers, and the like, are included within the definition. Both full length proteins and fragments thereof are encompassed by the definition. The terms also include postexpression modifications of the polypeptide, for example, glycosylation, acetylation, phosphorylation, hydroxylation, and the like. Furthermore, for purposes of the present invention, a “polypeptide” refers to a protein which includes modifications, such as deletions, additions and substitutions to the native sequence, so long as the protein maintains the desired activity. These modifications may be deliberate, as through site directed mutagenesis, or may be accidental, such as through mutations of hosts which produce the proteins or errors due to PCR amplification.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains biological activity, such as catalytic activity, ligand binding activity, or regulatory activity, as defined herein.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

As used herein, the terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof which is capable of exhibiting fluorescence in the detectable range. The term also includes fluorescent proteins and polypeptides.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as R-spondin antagonist activity (i.e., inhibiting activation of Wnt signaling by an R-spondin). In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, breast cancer, prostate cancer, lung cancer, ovarian cancer, testicular cancer, colon cancer, rectal cancer, pancreatic cancer, gastrointestinal cancer, hepatic cancer, endometrial cancer, leukemia, lymphoma, adrenal cancer, thyroid cancer, pituitary cancer, adrenocortical cancer, renal cancer, brain cancer (e.g., glioblastoma and astrocytoma), skin cancer (e.g., basal-cell cancer, squamous-cell cancer, and melanoma), head cancer, neck cancer, oral cavity cancer, tongue cancer, and esophageal cancer cancer.

An “effective amount” of an R-spondin antagonist (e.g., a RNF43 ECD-Fc fusion protein or a nucleic acid encoding such a fusion protein) is an amount sufficient to effect beneficial or desired results, such as an amount that inhibits activation of the Wnt signaling pathway, for example, by inhibiting one or more R-spondin proteins (e.g., R-spondin 1 (RSPO1), R-spondin 2 (RSPO2), R-spondin 3 (RSPO3), and R-spondin 4 (RSPO4)). An effective amount can be administered in one or more administrations, applications, or dosages.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “treatment” or “treating” as used herein refers to the ability to ameliorate, suppress, mitigate, or eliminate the clinical symptoms of a cancer responsive to inhibition of R-spondin and Wnt signaling.

The term “survival” as used herein means the time from the first dose of an R-spondin antagonist to the time of death.

By “therapeutically effective dose or amount” of an R-spondin antagonist (e.g., an RNF43 ECD-Fc fusion protein or a nucleic acid encoding an RNF43 ECD-Fc fusion protein) is intended an amount that, when administered as described herein, brings about a positive therapeutic response, such as an amount that has anti-tumor activity, inhibits metastasis, or increases survival of a subject treated for a cancer responsive to inhibition of R-spondin and Wnt signaling. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention relates to the discovery that R-spondin antagonists can be used for treating cancer associated with abnormal activation of Wnt signaling. In particular, the invention relates to compositions comprising R-spondin antagonists comprising a soluble extracellular domain of ring finger 43 (RNF43) or E3 ubiquitin ligase zinc and ring finger 3 (ZNRF3) and methods of treating cancer with such antagonists (see Example 1).

In order to further an understanding of the invention, a more detailed discussion is provided below regarding R-spondin antagonists and methods of treating cancer with such R-spondin antagonists.

A. R-Spondin Antagonists

In one aspect, the invention provides R-spondin antagonists that interfere with activation of the Wnt signaling pathway by binding to and inhibiting the activity of R-spondin proteins. At least four R-spondin proteins exist, including R-spondin 1 (RSPO1), R-spondin 2 (RSPO2), R-spondin 3 (RSPO3), and R-spondin 4 (RSPO4). Thus, in certain embodiments, one or more of the RSPO1, RSPO2, RSPO3, and RSPO4 proteins are inhibited. Preferably, at least RSPO2 and RSPO3 are inhibited by the R-spondin antagonist, and more preferably, all of the R-spondin proteins are inhibited. Inhibition may be complete or partial (i.e., all activity, some activity, or most activity is blocked by an inhibitor). For example, an R-spondin antagonist may reduce R-spondin activity by 70% to 100%, or any amount in this range, such as 70%, 72%, 74%, 76%, 78%, 80%, 82%, 84%, 86%, 88%, 90%, 92%, 94%, 96%, 98%, or 100% as compared to native or control levels. In certain embodiments, the R-spondin antagonist binds to at least one R-spondin protein with a dissociation constant (K_{D}) of less than 100 nM, more preferably, less than 1 nM, and most preferably, less than 10 pM.

In one embodiment, the R-spondin antagonist is a fusion protein (i.e., RNF43 ECD-Fc fusion protein) comprising a signal peptide (amino acids 1-23) and an extracellular domain (amino acids 24-192) of ring finger protein 43 (RNF43) covalently connected to an immunoglobulin Fc fragment. The foregoing numbering is relative to murine RNF43 (SEQ ID NO:4), and it is to be understood that the corresponding positions in RNF43 obtained from other species are also intended to be encompassed by the present invention.

RNF43 nucleic acid and protein sequences may be derived from any source. A number of RNF43 nucleic acid and protein sequences are known. Representative RNF43 sequences are presented in SEQ ID NO:1 (signal peptide), SEQ ID NO:2 (ECD), and SEQ ID NO:4 (entire RNF43), and additional representative sequences are listed in the National Center for Biotechnology Information (NCBI) database. See, for example, NCBI entries: Accession Nos. NM_001305544, NM_001305545, NM_017763, NG_042894, NM_172448, NM_001135921, XM_006983219, XM_015961792, XM_007429040, XM_015880934, XM_015817188, XM_015646883, XM_015646881, XM_015607484, XM_006924877, XM_006924876, XM_006756201, XM_006756202, XM_015539550, XM_015495419, XM_005219941, XM_005583828, XM_015383684, XM_015383683, XM_006640990, XM_003642379, XM_015251931, XM_006219108, XM_015119152, XM_015098454, XM_012170327, XM_014989054, XM_015031841, XM_015088435, XM_014959717, XM_014881490, XM_014881488, XM_014826904, XM_014826896, XM_014826886, XM_014826878, XM_014826870, XM_014826861, XM_014826856, XM_014741472, XM_014741466, XM_014741463, XM_014741458, XM_014741453, XM_014741451, XM_014741448, XM_006267924, XM_006154644, XM_014529848, XM_005858364, XM_014529841, XM_006126750, XM_006126749, XM_014556497, XM_014556496, XM_006181416, XM_014526439, XM_014486261, XM_005900646, XM_014469427, XM_014461677, XM_006095618, XM_014461676, XM_008970573, XM_008970572, XM_008970571, XM_003817383, XM_008970569, XM_008970568, XM_008970567, XM_005425150, XM_005493759, XM_014255106, XM_005439600, XM_014136583, XM_011524956, XM_011524955, XM_011524954, XM_006532701, XM_011248844, XM_006536449, XM_011251825, XM_006247094, XM_008768041, XM_008768040, XM_006247092, XM_008768039, XM_006247091, XM_006247090, XM_008768038, XM_009432917, XM_003315636, XM_009432916, XM_009432915, XM_009432914, XM_009432913, XM_009432912, XR 675406, XM_014116703, XM_014116702, XM_014116701, XM_014116700, XM_014116699, XM_014116698, and XM_014116697; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein comprising an RNF43 signal peptide and ECD, or a nucleic acid encoding such a fusion protein, as described herein.

The immunoglobulin Fc fragment may be derived from an IgG (e.g., IgG1, IgG2, IgG3, or IgG4), IgM, IgE, IgA or IgD, or a combination or hybrid thereof. In one embodiment, the Fc fragment is derived from an IgG2a immunoglobulin, wherein the fragment comprises the amino acid sequence of SEQ ID NO:3 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the fusion protein inhibits R-spondin activity.

The signal peptide and ECD of RNF43 and the immunoglobulin Fc fragment included in the fusion construct may be connected directly to each other by peptide bonds or may be separated by intervening amino acid sequences (i.e., linker). In certain embodiments, the fusion protein can be represented by the formula NH₂—SP-ECD-Fc-COOH or NH₂-A-SP-ECD-L-Fc-B—COOH, wherein: SP is an amino acid sequence of a RNF43 signal peptide; ECD is an amino acid sequence of a RNF43 extracellular domain; Fc is an amino acid sequence of an immunoglobulin Fc fragment; L is an optional linker amino acid sequence; A is an optional N-terminal amino acid sequence; and B is an optional C-terminal amino acid sequence. Multiple linkers can be used to connect the various polypeptides of the fusion. Where more than one linker is present in the fusion, the linkers can be the same or different.

Linker amino acid sequence(s) -L- will typically be short, e.g., 20 or fewer amino acids (i.e., 20, 19, 18, 17, 16, 15, 14, 13, 12, 11, 10, 9, 8, 7, 6, 5, 4, 3, 2, or 1). Examples include short peptide sequences which facilitate cloning, poly-glycine linkers (Glyₙ where n=2, 3, 4, 5, 6, 7, 8, 9, 10 or more), histidine tags (Hisₙ where n=3, 4, 5, 6, 7, 8, 9, 10 or more), linkers composed of glycine and serine residues, GSAT, SEG, and Z-EGFR linkers. Linkers may include restriction sites, which aid cloning and manipulation. Other suitable linker amino acid sequences will be apparent to those skilled in the art. (See e.g., Argos (1990) J. Mol. Biol. 211(4):943-958; Crasto et al. (2000) Protein Eng. 13:309-312; George et al. (2002) Protein Eng. 15:871-879; Arai et al. (2001) Protein Eng. 14:529-532; and the Registry of Standard Biological Parts (partsregistry.org/Protein_domains/Linker).

-A- is an optional N-terminal amino acid sequence. This will typically be short, e.g., 40 or fewer amino acids (i.e., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). Examples include leader sequences to direct protein localization, or short peptide sequences or tag sequences, which facilitate cloning or purification (e.g., a histidine tag Hisₙ where n=3, 4, 5, 6, 7, 8, 9, 10 or more). Other suitable N-terminal amino acid sequences will be apparent to those skilled in the art.

-B- is an optional C-terminal amino acid sequence. This will typically be short, e.g., 40 or fewer amino acids (i.e., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). Examples include sequences to direct protein localization, short peptide sequences or tag sequences, which facilitate cloning or purification (e.g., Hisₙ where n=3, 4, 5, 6, 7, 8, 9, 10 or more), or sequences which enhance protein stability. Other suitable C-terminal amino acid sequences will be apparent to those skilled in the art.

In certain embodiments, tag sequences are located at the N-terminus or C-terminus of the fusion protein. Exemplary tags that can be used in the practice of the invention include a His-tag, a Strep-tag, a TAP-tag, an S-tag, an SBP-tag, an Arg-tag, a calmodulin-binding peptide tag, a cellulose-binding domain tag, a DsbA tag, a c-myc tag, a glutathione S-transferase tag, a FLAG tag, a HAT-tag, a maltose-binding protein tag, a NusA tag, and a thioredoxin tag.

B. Production of R-Spondin Antagonists

R-spondin antagonists (e.g., RNF43 ECD-Fc fusion protein) can be prepared in any suitable manner (e.g., recombinant expression, purification from cell culture, chemical synthesis, etc.) and in various forms (e.g. native, fusions, labeled, lipidated, amidated, acetylated, etc.). RNF43 and Fc polypeptides include naturally-occurring polypeptides, recombinantly produced polypeptides, synthetically produced polypeptides, or polypeptides produced by a combination of these methods. Means for preparing peptides and polypeptides as well as fusion proteins are well understood in the art. Peptides, polypeptides, and fusion proteins are preferably prepared in substantially pure form (i.e. substantially free from other host cell or non-host cell proteins).

In one embodiment, the R-spondin antagonist is generated using recombinant techniques. One of skill in the art can readily determine nucleotide sequences that encode the desired peptides, polypeptides, or fusion proteins using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding peptides, polypeptides, or fusion proteins can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding peptides or polypeptides that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified peptides can be generated by creating expression constructs which operably link, in various combinations, polynucleotides encoding peptides having deletions or mutations therein. Numerous cloning vectors are known to those of skill in the art, and the selection of an appropriate cloning vector is a matter of choice. Examples of recombinant DNA vectors for cloning and host cells which they can transform include the bacteriophage (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), 0.161 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce R-spondin antagonists described herein. Generally, such systems use virus-based vectors to transfect plant cells with heterologous genes. For a description of such systems, see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fusion proteins of the present invention are produced by growing host cells transformed by an expression vector described above under conditions whereby the protein of interest is expressed. The selection of the appropriate growth conditions is within the skill of the art.

In one embodiment, the transformed cells secrete the peptide or polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted peptide or polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant peptides or polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced peptides or polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular peptides or polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the peptides or polypeptides can be further purified using conventional techniques well known in the art, such as by any of the techniques described above.

R-spondin antagonists can be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final peptide or polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethyl styrene copolymers, divinylbenzene-chloromethyl styrene copolymers and divinylbenzene-b enzhydrylaminopolystyrene copolymers.

R-spondin antagonists can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

C. Nucleic Acids Encoding R-Spondin Antagonists

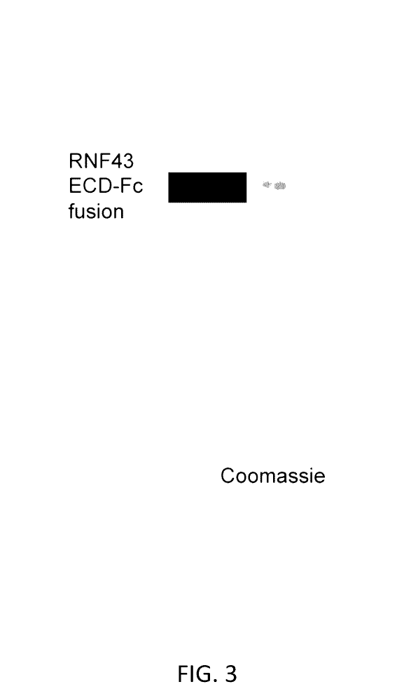

Nucleic acids encoding R-spondin antagonists, such as an RNF43 ECD-Fc fusion protein can be used, for example, to treat cancers responsive to inhibition of R-spondin and Wnt signaling. Nucleic acids described herein can be inserted into an expression vector to create an expression cassette capable of producing the R-spondin antagonist in a suitable host cell. The ability of constructs to produce the R-spondin antagonist can be empirically determined (e.g., see Example 1 and FIG. 3 describing detection of a 48 kDa band for the RNF43 ECD-Fc fusion protein by SDS-PAGE).

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding the R-spondin antagonist can be administered to a subject using standard gene delivery protocols. Methods for gene delivery are known in the art. See, e.g., U.S. Pat. Nos. 5,399,346, 5,580,859, 5,589,466. Genes can be delivered either directly to a vertebrate subject or, alternatively, delivered ex vivo, to cells derived from the subject and the cells reimplanted in the subject.

A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the R-spondin antagonist of interest include those derived from the pox family of viruses, including vaccinia virus and avian poxvirus. By way of example, vaccinia virus recombinants expressing the R-spondin antagonist can be constructed as follows. The DNA encoding the particular R-spondin antagonist or RNF43 ECD-Fc fusion protein coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia based infection/transfection system can be conveniently used to provide for inducible, transient expression of the coding sequences of interest (for example, a RNF43 ECD-Fc expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Feigner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Feigner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering synthetic expression cassettes of the present invention. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors carrying a synthetic expression cassette of the present invention are formulated into compositions for delivery to a vertebrate subject. These compositions may either be prophylactic (prevent cancer progression of cells or tissues with aberrant activation of Wnt signaling) or therapeutic (to treat cancer). The compositions will comprise a “therapeutically effective amount” of the nucleic acid of interest such that an amount of the R-spondin antagonist (e.g., RNF43 ECD-Fc fusion protein) can be produced in vivo so that R-spondin and concomitantly Wnt signaling are inhibited in the individual to which it is administered. The exact amount necessary will vary depending on the subject being treated; the age and general condition of the subject to be treated; the degree of protection desired; the severity of the condition being treated; the particular R-spondin antagonist produced and its mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions of the invention can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject or a gene gun, such as the Accell gene delivery system (PowderMed Ltd, Oxford, England).

D. Pharmaceutical Compositions

An R-spondin antagonist (e.g., a RNF43 ECD-Fc fusion protein or a nucleic acid encoding such a fusion protein) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the R-spondin antagonist (e.g., fusion protein, or nucleic acid encoding the fusion protein), or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfate, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the R-spondin antagonist (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising an R-spondin antagonist (e.g., a RNF43 ECD-Fc fusion protein or a nucleic acid encoding such a fusion protein) are in unit dosage form, meaning an amount of a conjugate or composition of the invention appropriate for a single dose, in a premeasured or pre-packaged form.

The compositions herein may optionally include one or more additional agents, such as other drugs for treating cancer or other medications used to treat a subject for a condition or disease. Compounded preparations may include an R-spondin antagonist (e.g., a RNF43 ECD-Fc fusion protein or a nucleic acid encoding such a fusion protein) and one or more drugs for treating cancer, such as, but not limited to, chemotherapy, immunotherapy, biologic or targeted therapy agents. Alternatively, such agents can be contained in a separate composition from the composition comprising an R-spondin antagonist (e.g., a RNF43 ECD-Fc fusion protein or a nucleic acid encoding such a fusion protein) and co-administered concurrently, before, or after the composition comprising an R-spondin antagonist of the invention.

E. Administration

The methods of the invention can be used for treating a subject for any cancer responsive to inhibition of R-spondin and Wnt signaling. Thus, R-spondin antagonists of the invention can be used to treat, for example, neoplasia, tumors, or cancers, including benign, malignant, metastatic and non-metastatic types, including any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. Furthermore, the methods described herein can be used to treat various types of cancer, including, but not limited to, breast cancer, prostate cancer, lung cancer, ovarian cancer, testicular cancer, colon cancer, rectal cancer, pancreatic cancer, gastrointestinal cancer, hepatic cancer, endometrial cancer, leukemia, lymphoma, adrenal cancer, thyroid cancer, pituitary cancer, adrenocortical cancer, renal cancer, brain cancer (e.g., glioblastoma and astrocytoma), skin cancer (e.g., basal-cell cancer, squamous-cell cancer, and melanoma), head cancer, neck cancer, oral cavity cancer, tongue cancer, and esophageal cancer cancer, or any other cancer associated with aberrant activation of Wnt signaling.

At least one therapeutically effective cycle of treatment with an R-spondin antagonist (e.g., RNF43 ECD-Fc fusion protein or a nucleic acid encoding an R-spondin antagonist) will be administered to a subject for treatment of cancer. By “therapeutically effective dose or amount” of an R-spondin antagonist is intended an amount that when administered brings about a positive therapeutic response with respect to treatment of an individual for cancer. Of particular interest is an amount of an R-spondin antagonist that provides an anti-tumor effect, as defined herein. By “positive therapeutic response” is intended the individual undergoing the treatment according to the invention exhibits an improvement in one or more symptoms of the cancer for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In certain embodiments, multiple therapeutically effective doses of compositions comprising an R-spondin antagonist (e.g., RNF43 ECD-Fc fusion protein), or a nucleic acid encoding an R-spondin antagonist, and/or one or more other therapeutic agents, such as other drugs for treating cancer, or other medications will be administered. The compositions of the present invention are typically, although not necessarily, administered orally, via injection (subcutaneously, intravenously, or intramuscularly), by infusion, or locally. Additional modes of administration are also contemplated, such as intraperitoneal, pulmonary, nasal, topical, transdermal, intralesion, intraparenchymatous, rectal, transdermal, transmucosal, intrathecal, pericardial, intra-arterial, intraocular, and so forth.

The preparations according to the invention are also suitable for local treatment. In a particular embodiment, a composition of the invention is used for localized delivery of an R-spondin antagonist, for example, for the treatment of cancer. For example, compositions may be administered directly into a tumor or cancerous cells. The particular preparation and appropriate method of administration are chosen to target the an R-spondin antagonist to the site of a tumor or cancerous cells or site of aberrant activation of Wnt signaling.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising an R-spondin antagonist and other agents may be administered using the same or different routes of administration in accordance with any medically acceptable method known in the art.

In another embodiment, the pharmaceutical compositions comprising an R-spondin antagonist and/or other agents are administered prophylactically, e.g., to prevent cancer progression of cells or tissues with aberrant activation of Wnt signaling. Such prophylactic uses will be of particular value for subjects with a potentially precancerous or premalignant condition (e.g., dysplasia or benign neoplasia exhibiting aberrant activation of Wnt signaling), or who have a genetic predisposition to developing cancer.

In another embodiment of the invention, the pharmaceutical compositions comprising an R-spondin antagonist and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising an R-spondin antagonist as provided herein to a patient suffering from a cancer that is responsive to treatment with an R-spondin antagonist contained in the conjugate or composition. The method comprises administering, via any of the herein described modes, a therapeutically effective amount of the conjugate or drug delivery system, preferably provided as part of a pharmaceutical composition. The method of administering may be used to treat any cancer that is responsive to treatment with an R-spondin antagonist. More specifically, the compositions herein are effective in treating a cancer responsive to inhibition of R-spondin and Wnt signaling.

Those of ordinary skill in the art will appreciate which conditions a specific R-spondin antagonist can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of an R-spondin antagonist inhibitor daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific R-spondin antagonist and the magnitude or effect on R-spondin inhibition desired and the route of administration.

A purified R-spondin antagonist (again, preferably provided as part of a pharmaceutical preparation) can be administered alone or in combination with one or more other therapeutic agents, such as chemotherapy, immunotherapy, biologic or targeted therapy agents, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

An R-spondin antagonist can be administered prior to, concurrent with, or subsequent to other agents. If provided at the same time as other agents, the R-spondin antagonist can be provided in the same or in a different composition. Thus, the R-spondin antagonist and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising an R-spondin antagonist and a dose of a pharmaceutical composition comprising at least one other agent, such as another drug for treating cancer, which in combination comprise a therapeutically effective dose, according to a particular dosing regimen. Similarly, the R-spondin antagonist and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, a subject may receive one or more additional treatment periods with the R-spondin antagonist. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

Additionally, treatment with an R-spondin antagonist may be combined with any other medical treatment for cancer, such as, but not limited to, surgery, radiation therapy, chemotherapy, hormonal therapy, immunotherapy, or molecularly targeted or biologic therapy. Any combination of these other medical treatment methods with an R-spondin antagonist may be used to effectively treat cancer in a subject.

For example, treatment with an R-spondin antagonist may be combined with chemotherapy with one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

In another example, treatment with an R-spondin antagonist may be combined with targeted therapy with one or more small molecule inhibitors or monoclonal antibodies such as, but not limited to, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

In a further example, treatment with an R-spondin antagonist may be combined with immunotherapy, including, but not limited to, using any of the following: a cancer vaccine (e.g., Sipuleucel-T), antibody therapy (e.g., Alemtuzumab, Ipilimumab, Ofatumumab, Nivolumab, Pembrolizumab, or Rituximab), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy.

F. Kits

The invention also provides kits comprising one or more containers holding compositions comprising at least one R-spondin antagonist (e.g., a RNF43 ECD-Fc fusion protein) or nucleic acid encoding a an R-spondin antagonist), and optionally one or more other drugs for treating cancer. Compositions can be in liquid form or can be lyophilized, as can individual peptides, polypeptides, or nucleic acids. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating cancer. The package insert can be an unapproved draft package insert or can be a package insert approved by the Food and Drug Administration (FDA) or other regulatory body.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Engineered R-Spondin Antagonists

Introduction

Wnt pathway activation has been implicated in the pathological progression of colon cancer. R-spondin proteins are potent agonists of the Wnt pathway. Thus, we proposed that inhibition of R-spondin proteins in order to inhibit signaling of the Wnt pathway would be an effective strategy for treating colon cancer. Selective inhibition of R-spondin is complicated by the existence of 4 R-spondin family members and their functional redundancy.

The R-spondins are known to be ligands for the RNF43 and ZNRF3 receptors. Here we report the discovery that soluble ectodomains of the RNF43 and ZNRF3 R-spondin receptors interfere with autocrine R-spondin signaling by binding to and neutralizing R-spondin proteins.

Results

We constructed an R-spondin antagonist comprising a soluble extracellular domain (ECD) of RNF43 comprising amino acids 1-192 of murine RNF43 (23 amino acid signal peptide (SEQ ID NO:1)+169 amino acid mature coding sequence (SEQ ID NO:2)) fused to a 236 amino acid mouse IgG2a immunoglobulin Fc fragment (SEQ ID NO:3).

Native signal peptide 23 aa
(SEQ ID NO: 1)
MSGGHQLQLA VLWPWLLMAT LHA
RNF43 ECD 169 aa
(SEQ ID NO: 2)
G F G H T G R V L A A A V E S E R S A E Q K A V I
R V I P L K M D P T G K L N L T L E G V F A G V A
E V T P A E G K L M Q S H P L Y L C N A S D D D N
L E P G F I S I V K L E S P R R A P R P C L S L A
S K A R M A G E R G A N A V L F D I T E D R S A A
E Q L Q Q P L G L T K P V V L I W G S D A A K L M
E F V Y K N R K A Y V W I E L K E P P
Mouse IgG Fc 236 aa
(SEQ ID NO: 3)
R R L E P R G P T I K P C P P C K C P A P N L L G
G P S V F I F P P K I K D V L M I S L S P I V T C
V V V D V S E D D P D V Q I S W F V N N V E V H T
A Q T Q T H R E D Y N S T L R V V S A L P I Q H Q
D W M S G K E F K C K V N N K D L P A P I E R T I
S K P K G S V R A P Q V Y V L P P P E E E M T K K
Q V T L T C M V T D F M P E D I Y V E W T N N G K
T E L N Y K N T E P V L D S D G S Y F M Y S K L R
V E K K N W V E R N S Y S C S V V H E G L H N H H
T T K S F S R T P G K

The RNF43 ECD binds R-spondin and prevents it from binding to RNF43/ZNRF3. Thus the RNF43/ZNRF3 degradation of Frizzled and LRP Wnt receptors is no longer suppressed. Accordingly, RNF43/ZNRF3 degradation of Frizzled and LRP Wnt receptors occurs in an unrestricted fashion, and Frizzed LRP are decreased in abundance with an overall repression of Wnt signaling.

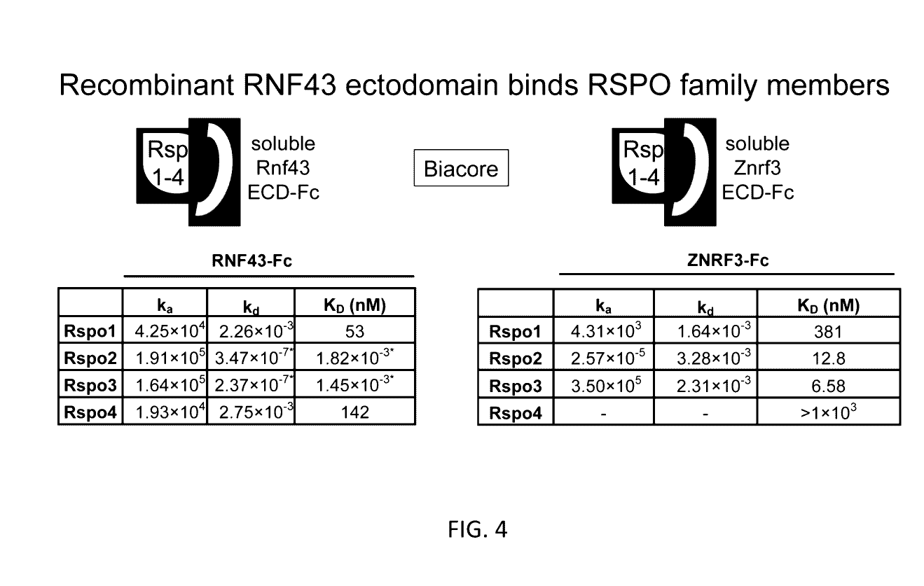

The RNF43 ECD-Fc fusion construct was cloned into a CMV expression vector and transfected into 293T cells where the soluble RNF43-Fc ECD fusion protein eluted as a single species of 48 kDa after purification and elution from protein A agarose (FIG. 3). Further, the purified soluble RNF43-Fc ECD fusion protein bound to recombinant R-spondin in Biacore analyses, with a Kd of 53 nM and 142 nM for human RSPO1 and RSPO4, respectively) but extremely tight binding to human RSPO2 and RSPO3 (1.82 pM and 1.45 pM, respectively). The affinity of the RNF43-Fc ECD for RSPO1-4 was much stronger than an equivalent ZNRF3-Fc ECD fusion that we tested in parallel, about 7000-fold stronger for RSPO2 and about 4500-fold stronger for RSPO3 (FIG. 4).

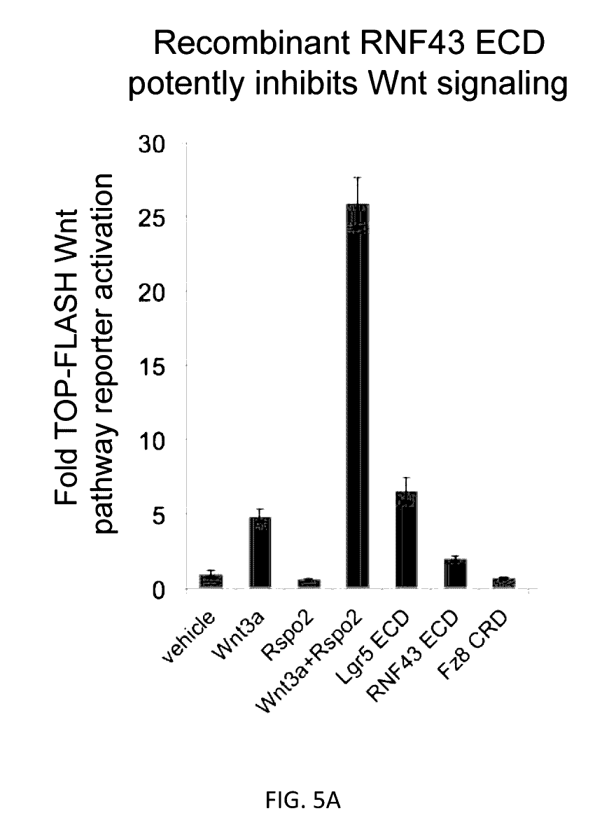

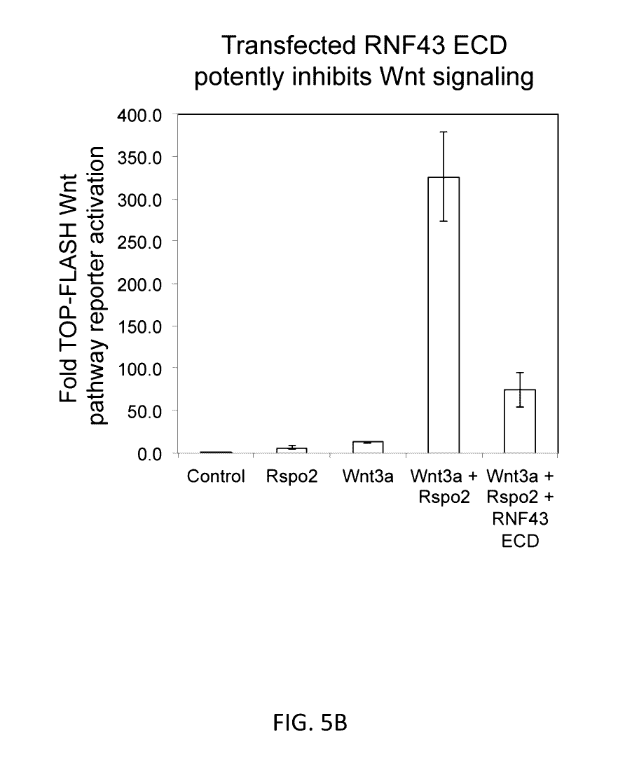

We further demonstrated that the recombinant RNF43-Fc ECD fusion protein potently neutralized the ability of RSPO proteins to activate Wnt signaling in a TOP-FLASH Wnt reporter assay (FIG. 5, left). Similar results were obtained when the RNF43-Fc ECD fusion protein was transfected (FIG. 5, right).

We next examined the ability of the RNF43-Fc ECD fusion protein to inhibit tumor growth, using a colorectal cancer model bearing the RSPO3 translocation that had been identified in human patients. Because there are no current experimental models of RSpo-dependent colon cancer, we created an organoid model. We have pioneered methods for the engineering of colon cancer from primary colon organoid cultures in an air-liquid interface 2 and can fully convert normal colon to adenocarcinoma in vitro by viral introduction of >4 oncogenes/tumor suppressors, even allowing in vivo transplantable cancer.

The RSPO3 translocation in human colorectal cancer patients essentially overexpresses full-length RSPO3 under heterologous promoters, and occurs with combinations of mutant Kras, p53, BRAF and/or PIK3CA. While >85% of CRCs harbor APC mutations with constitutive Wnt signaling, EIF3E-RSPO2 and PTPRK-RSPO3 translocations occur in ˜10% of human CRC, with >80-fold upregulation of expression 14. These translocations are mutually exclusive with APC mutation, consistent with an alternative mechanism for Wnt activation in CRC. The autocrine RSPO signaling induced by these translocations are an unusual therapeutic opportunity for treatment with extracellular RSPO antagonists, such as our RNF43 ECD, especially because the RNF43 ECD is both highly specific for RSPO2 and RSPO3 which are precisely the RSPO family members that are translocated in human colon cancer. Further, the RNF43 ECD has 4500-7000× higher affinity for RSPO2 and RSPO3 than does the ECD of ZNRF3, the RNF43-related family member.

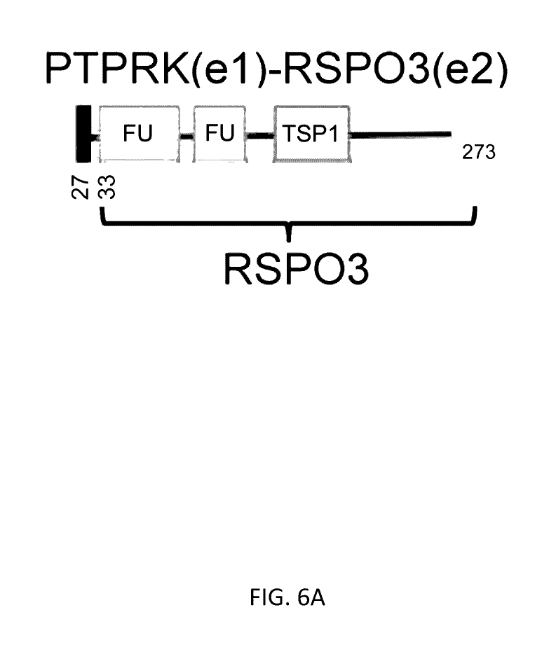

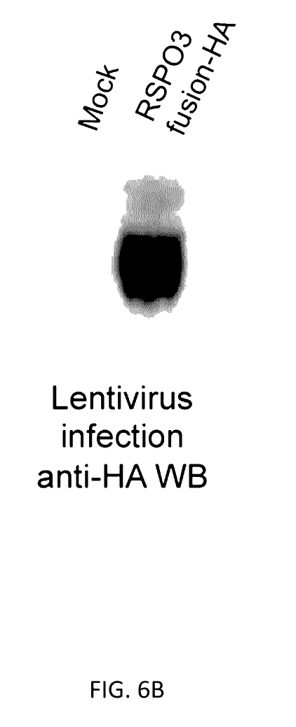

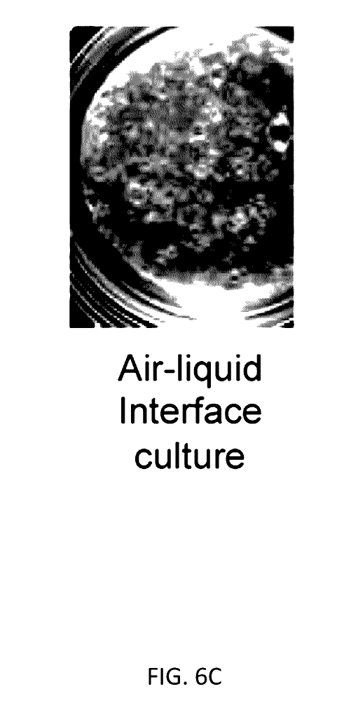

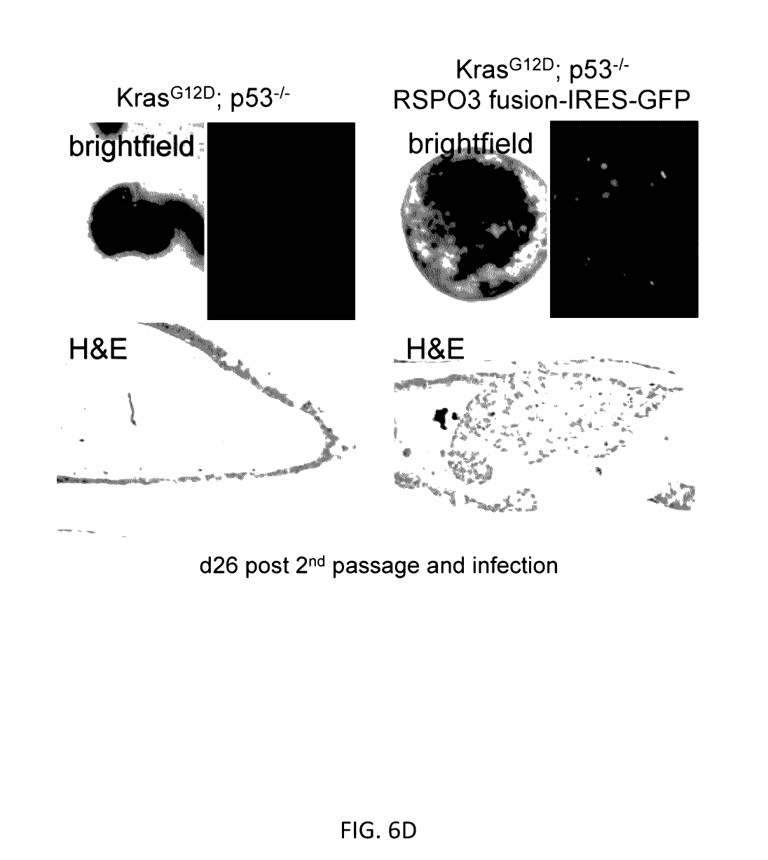

Using our published primary air-liquid interface organoid method we created primary colon organoids from lox-stop-lox KRASG12D; p53flox/flox mice and infected with adenovirus Cre to create KRASG12D; p53−/− colon organoids. Lentivirus encoding the RSPO3 fusion (PTPRK-RSPO3) oncogene-HA-IRES-GFP both strongly expressed secreted RSPO3 and induced severe dysplasia in organoid culture (FIG. 6).

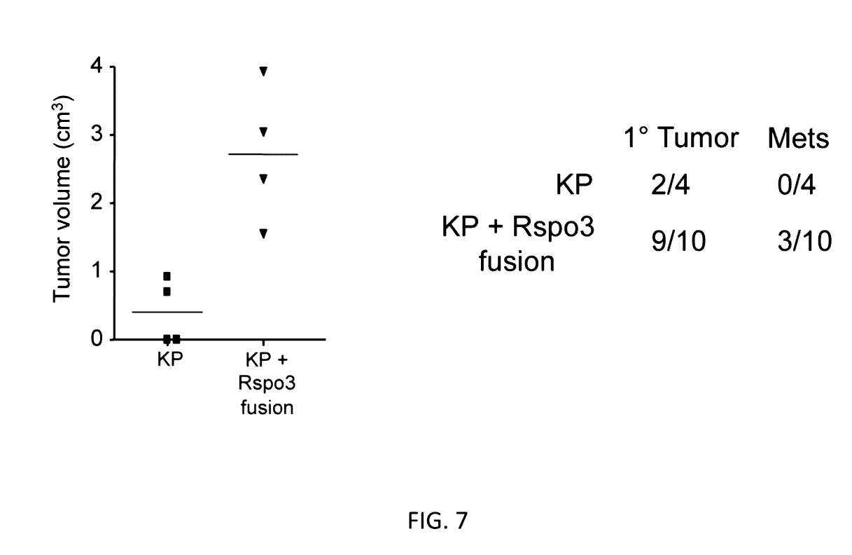

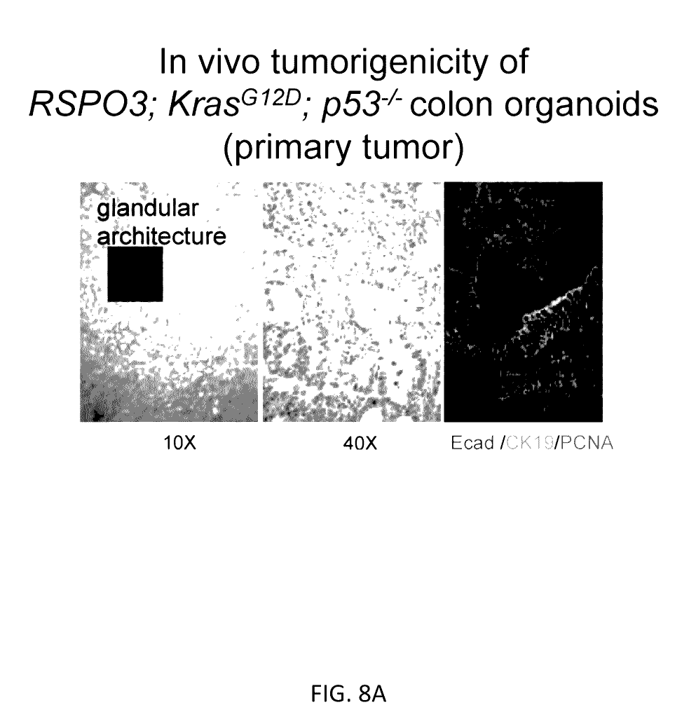

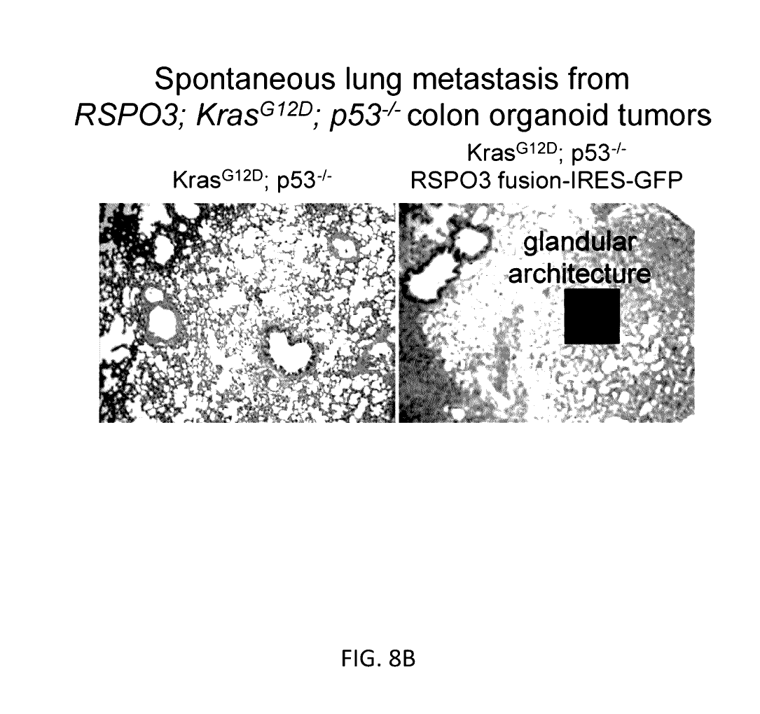

We then implanted the KRAS^{G12D}; p53^{-/-} colon organoids with or without lentiviral expression of the RSPO3 fusion transcript subcutaneously (s.c.) into NOG mice. Notably, RSPO3 strongly promoted the s.c. growth of the KRAS^{G12D}; p53^{-/-} colon organoids with larger tumor size and higher take rate observed (FIG. 7). Further, RSPO3 also induced spontaneous lung metastasis of the KRAS^{G12D}; p53^{-/-} colon organoids from the s.c. primary tumors (FIG. 7). Histology of the primary and metastatic tumors revealed a common glandular histology (FIG. 8).

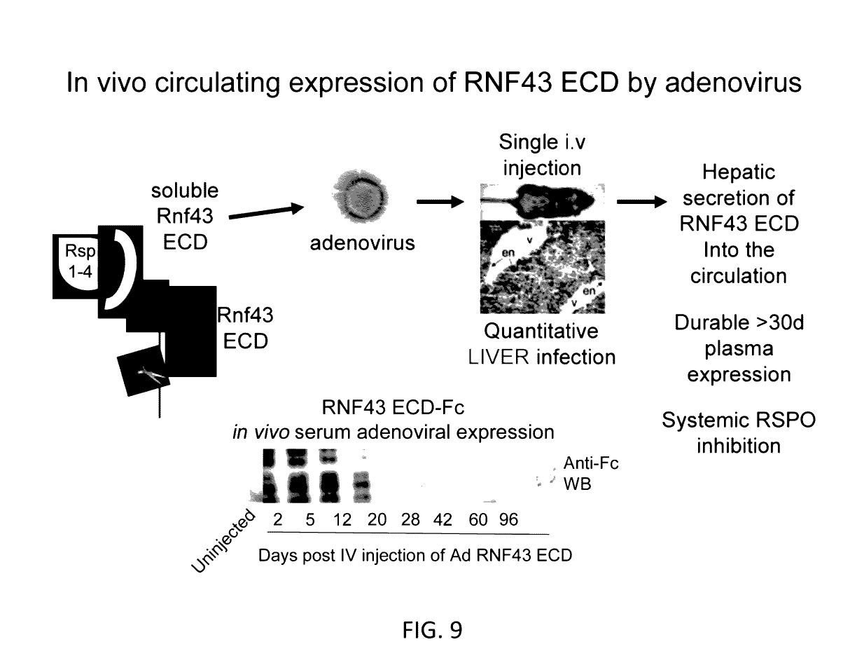

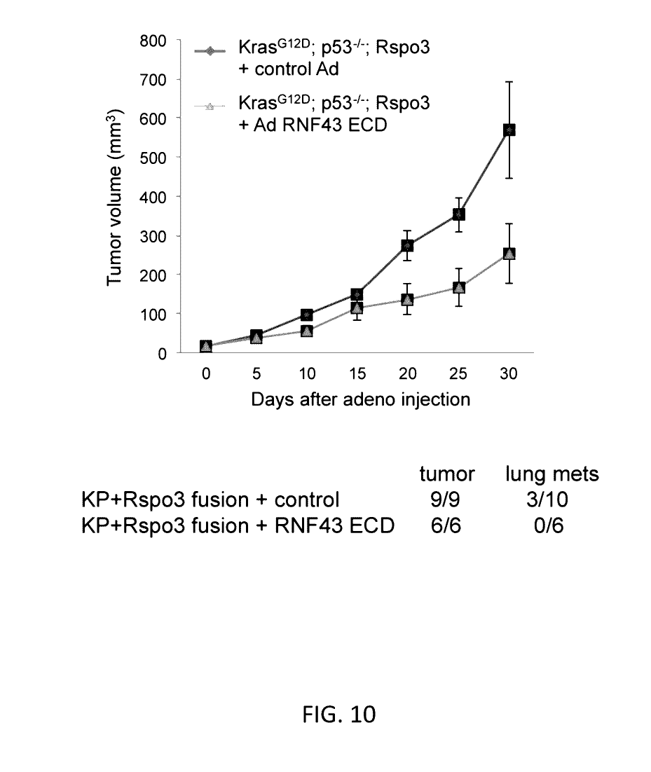

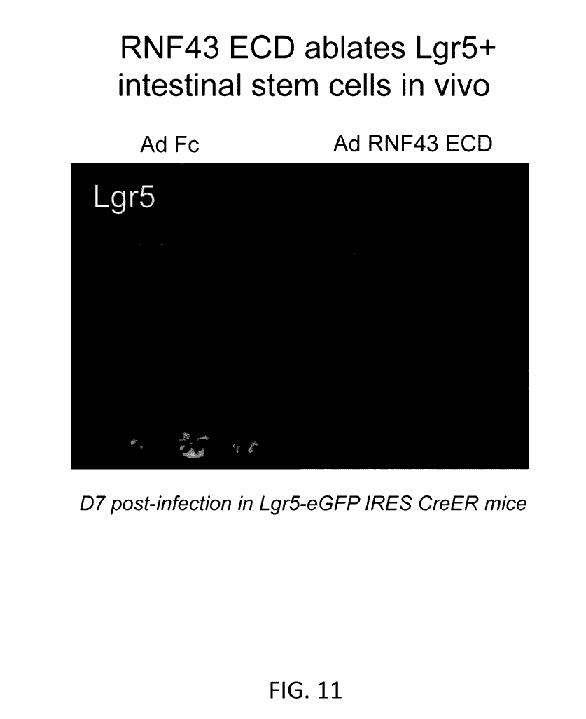

Next, we tested the ability of the RNF43-Fc ECD fusion protein to inhibit tumorigenesis in the KRAS^{G12D}; p53^{-/-}; RSPO3 fusion colon organoid tumor model. For convenience we delivered the RNF43-Fc ECD fusion protein by adenovirus to tumor bearing NOG mice that had been previously implanted s.c. with KRAS^{G12D}; p53^{-/-} colon organoids that had been lentivirally transduced with the RSPO3 fusion gene. Under these conditions, single intravenous (i.v.) injection of the adenovirus expressing the RNF43-Fc ECD gave rise to liver transduction and hepatocyte secretion of the RNF43-Fc ECD into the circulation for >96 days (FIG. 9). Importantly, the adenovirus expressing the RNF43 ECD-Fc fusion strongly inhibited both tumor growth and spontaneous lung metastasis of KRAS^{G12D}; p53^{-/-}; RSPO3 fusion tumors relative to a control adenovirus (FIG. 10). Notably, despite persistent >96 day adenoviral expression of the RNF43-Fc ECD fusion in the circulation, the recipient mice were normal without physiologic compromise. However, we did note an essentially complete suppression of Lgr5+ intestinal stem cells, consistent with a role for RSPO proteins in maintenance of this stem cell population as well as published literature indicating that Lgr5+ intestinal stem cells are not essential for life.

Although preferred embodiments of the subject invention have been described in some detail, it is understood that obvious variations can be made without departing from the spirit and the scope of the invention as defined herein.

What is claimed is:

1. An R-spondin antagonist comprising a soluble extracellular domain of ring finger 43 (RNF43) or E3 ubiquitin ligase zinc and ring finger 3 (ZNRF3).

2. The R-spondin antagonist of claim 1, wherein the antagonist is a fusion protein comprising an immunoglobulin Fc domain covalently linked to the soluble extracellular domain of the RNF43 or ZNRF3.

3. The R-spondin antagonist of claim 2, wherein the fusion protein comprises residues corresponding to amino acids 1 to 192 of the RNF43 numbered relative to the reference sequence of SEQ ID NO:4.

4. The R-spondin antagonist of claim 2, wherein the immunoglobulin Fc domain is from an immunoglobulin G (IgG) selected from the group consisting of IgG1, IgG2, IgG3, and IgG4.

5. The R-spondin antagonist of claim 4, wherein the Fc domain comprises:
a) a polypeptide comprising the amino acid sequence of SEQ ID NO:3; or
b) a polypeptide comprising an amino acid sequence having at least 90% identity to the amino acid sequence of SEQ ID NO:3, wherein the R-spondin antagonist inhibits activation of Wnt signaling by an R-spondin.

6. The R-spondin antagonist of claim 1, wherein the soluble extracellular domain of RNF43 comprises:
a) a polypeptide comprising the amino acid sequence of SEQ ID NO:2; or
b) a polypeptide comprising an amino acid sequence having at least 90% identity to the amino acid sequence of SEQ ID NO:2, wherein the R-spondin antagonist inhibits activation of Wnt signaling by an R-spondin.

7. The R-spondin antagonist of claim 1, further comprising a RNF43 signal peptide.

8. The R-spondin antagonist of claim 7, wherein the signal peptide comprises the amino acid sequence of SEQ ID NO:1.

9. The R-spondin antagonist of claim 1, wherein the R-spondin antagonist binds to an R-spondin selected from the group consisting of R-spondin 1, R-spondin 2, R-spondin 3, and R-spondin 4.

10. The R-spondin antagonist of claim 1, wherein the R-spondin antagonist binds to R-spondin 1, R-spondin 2, R-spondin 3, and R-spondin 4.

11. The R-spondin antagonist of claim 1, wherein the R-spondin antagonist binds to a human R-spondin.

12. The R-spondin antagonist of claim 1, wherein the R-spondin antagonist binds to at least one R-spondin with a dissociation constant (K_{D}) of less than 10 pM.

13. A polynucleotide encoding the R-spondin antagonist of claim 1.

14. A recombinant polynucleotide comprising the polynucleotide of claim 13 operably linked to a promoter.

15. The recombinant polynucleotide of claim 14, wherein the recombinant polynucleotide comprises a plasmid or a viral vector.

16. The recombinant polynucleotide of claim 15, wherein the viral vector is an adenoviral expression vector or a cytomegalovirus (CMV) expression vector.

17. A host cell comprising the recombinant polynucleotide of claim 14.

18. The host cell of claim 17, wherein the host cell is a mammalian cell.

19. The host cell of claim 18, wherein the host cell is a human cell.

20. A method for producing an R-spondin antagonist, the method comprising:
a) transforming a host cell with the recombinant polynucleotide of claim 14;
b) culturing the transformed host cell under conditions whereby the R-spondin antagonist is expressed; and
c) isolating the R-spondin antagonist from the host cell.

21. A kit comprising the R-spondin antagonist of claim 1.

22. The kit of claim 21, wherein the R-spondin antagonist is a fusion protein comprising an immunoglobulin Fc domain covalently linked to the soluble extracellular domain of the RNF43 or ZNRF3.

23. The kit of claim 22, wherein the fusion protein comprises residues corresponding to amino acids 1 to 192 of the RNF43 numbered relative to the reference sequence of SEQ ID NO:4.

24. A kit comprising the recombinant polynucleotide of claim 14.

25. A method of treating a subject for cancer, the method comprising administering a therapeutically effective amount of the R-spondin antagonist of claim 1 to the subject.

26. The method of claim 25, wherein the cancer is colon cancer.

27. The method of claim 25, wherein treatment increases the expected survival time of the subject.

28. The method of claim 25, wherein multiple therapeutically effective doses of the R-spondin antagonist are administered to said subject.

29. The method of claim 28, wherein multiple cycles of treatment are administered to said subject for a time period sufficient to effect at least a partial tumor response.

30. The method of claim 29, wherein multiple cycles of treatment are administered to said subject for a time period sufficient to effect a complete tumor response.

31. The method of claim 25, wherein treatment inhibits metastasis in the subject

32. A method of treating a subject for cancer, the method comprising administering a therapeutically effective amount of the recombinant polynucleotide of claim 14 to the subject.

33. The method of claim 32, wherein the recombinant polynucleotide comprises a viral vector.

34. The method of claim 33, wherein the viral vector is an adenoviral expression vector.

35. The method of claim 32, wherein the recombinant polynucleotide is administered intravenously.

36. The method of claim 32, wherein the cancer is colon cancer.

37. The method of claim 32, wherein treatment increases the expected survival time of the subject.

38. The method of claim 32, wherein treatment inhibits metastasis in the subject

39. The method of claim 32, wherein treatment results in at least a partial tumor response.


SEQUENCE LISTING (4 sequences)

SEQ ID NO: 1 - protein, 23 aa, Mus musculus
MSGGHQLQLAVLWPWLLMATLHA

SEQ ID NO: 2 - protein, 169 aa, Mus musculus
GFGHTGRVLAAAVESERSAEQKAVIRVIPLKMDPTGKLNLTLEGVFAGVAEVTPAEGKLMQSHPLYLCNASDDDNLEPGFISIVKLESPRRAPRPCLSLASKARMAGERGANAVLFDITEDRSAAEQLQQPLGLTKPVVLIWGSDAAKLMEFVYKNRKAYVWIELKEPP

SEQ ID NO: 3 - protein, 236 aa, Mus musculus
RRLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 784 aa, Mus musculus
  ref <308>: DATABASE ACCESSION NUMBER: NCBI/NP_766036
  ref <309>: DATABASE ENTRY DATE: 2015-02-15
  ref <313>: RELEVANT RESIDUES IN SEQ ID NO: (1)..(784)
MSGGHQLQLAVLWPWLLMATLHAGFGHTGRVLAAAVESERSAEQKAVIRVIPLKMDPTGKLNLTLEGVFAGVAEVTPAEGKLMQSHPLYLCNASDDDNLEPGFISIVKLESPRRAPRPCLSLASKARMAGERGANAVLFDITEDRSAAEQLQQPLGLTKPVVLIWGSDAAKLMEFVYKNRKAYVWIELKEPPAGANYDVWILLTVVGTVFVIILASVLRIRCRPHHSRPDPLQQRTARAISQLATRRYQAGCRRARAEWPDSGSSCSSTPVCAICLEEFSEGQELRVISCLHEFHRTCVDPWLYQHRTCPLCMFNIVEGDSFSQAPAASPSYQEPGRRLHLIRQHPGHAHYHLPSAYLLGPSRTSVARTPRPRPFLPSQEPSMGSRHQRLPRTSHLRAPEEQQHLAVSPHPYAQGWGLNRLRCTSQHPAACPVALRRARPHESSGSGESYCTERSGYLADGPASDSSSGPCHGSSSDSVVNCTDVSLQGIHGSSSTFRSSLSSDFDPLVYCSPEGDLQGKGIQPSVTSRPRSLDSVVPRGETQVSSHIHYHRHRHHHYKRQFQWHGRKPGPETGIPQSMPAASHTQLEPSLPDQQLITPNPTASSMLPNPQRPRALTEPAPGLAEASSPSPSPKPNPSGLLNLQKSSLTVRHPHRKRRGGPSEPLPTSLPPDLTVHTACPVFPHYSPRLAYPWPPEVHPLMFRPPGPDRRLLHEVPGPCYSSSQPVWLYLNPCQPLGPCLPGEGHSKWTFDSPEGRRCPYSHCQVLPAQPGSEEELEELCEQAV